(19) United States
(12) Patent Application Publication
(10) Pub. No.: US 2020/0223792 A1
(43) Pub. Date: Jul. 16, 2020

(54) REGULATOR FOR THE PRODUCTION OF ALKANE SULFONIC ACIDS

(71) Applicant: BASF SE, Ludwigshafen am Rhein (DE)

(72) Inventor: Timo OTT, Duisburg (DE)

(73) Assignee: BASF SE, Ludwigshafen am Rhein (DE)

(21) Appl. No.: 16/627,009

(22) PCT Filed: Jun. 28, 2018
(86) PCT No.: PCT/EP2018/067385

§ 371 Date: Dec. 27, 2019

(30) Foreign Application Priority Data
Jun. 30, 2017 (EP) 17178926.6

(51) Int. Cl. C07C 303/22

(52) U.S. Cl. CPC C07C 303/22

(57) ABSTRACT

The present invention relates to a process for the production of alkane sulfonic acid with a regulator as well as two specific compounds which can be used as regulator in a respective process.

The present invention relates to a process for the production of alkane sulfonic acid with a regulator as well as to specific compounds which can be used as regulator in a respective process.

Alkane sulfonic acids are organic acids that can reach a similar acids strength as that of inorganic mineral acids, for example sulfuric acids. However, in contrast to usual mineral acids such as sulfuric or nitric acids, the sulfonic acids are non-oxidizing and do not give off vapors that are harmful to health, as can be observes with hydrochloric or nitric acids. The structurally simplest representative of alkane sulfonic acids is methane sulfonic acids. U.S. Pat. No. 2,493,038 describes the preparation of methane sulfonic acid from SO₃ and methane. US 2005/0070614 describes further methods for preparing methane sulfonic acid, and its application.

WO 2007/136425 A2 discloses the use of di(methanesulfonyl) peroxide (DMSP). Other initiator compounds being described in literature, for example peroxide acids as well as their salts, for example in WO 2004/041399 A2, or a highly effective asymmetric peroxide compound as disclosed in WO 2015/071455 A1.

Comparing DMSP and peroxodisulfuric acid (Marshall's acid) as initiator, it can be stated that DMSP leads to high selectivity in view of the formation of side products whereas Marshall's acid is very reactive but very little selective. The initiator as disclosed in WO 2015/071455 A1 combines the selectivity of DMSP with the reactivity of Marshall's acid.

The development of different initiators for the preparation of alkane sulfonic acids is time-consuming. There is therefore a need for a regulator which enables to regulate the reaction between an alkane and SO₃ to be either fast or more selective in view of the formation of alkane sulfonic acids, depending on the needs of the producer. For the reaction, the same initiator should be used, only the addition of the regulator should help to either enhance the reactivity or the selectivity or even both.

Surprisingly it has been found that a process for the production of alkane sulfonic acids from an alkane and SO₃ can be regulated by a transition metal or metal or metalloid of any of groups 3 to 14 of the periodic table, or a mixtures of two or more of those, wherein the transition metal or metal or metalloid is present in its elemental form, as salt, or as oxide.

The present invention thus also discloses the use of a transition metal or metal or metalloid of any of groups 3 to 14 of the periodic table, or mixtures of two or more of those, wherein the transition metal or metal or metalloid is present in its elemental form or as salt or as oxide as regulator in the production of alkane sulfonic acids form an alkane and SO₃.

The process according to the present invention allows for a controlled alkane sulfonation, especially methane sulfonation.

The regulator of the present invention might be any transition metal, metal or metalloid of any of groups 3 to 14 of the periodic table. Such transition metal, metal or metalloid might be used in its elemental form. It might also be used as salt or as oxide. Further, mixtures of two or more of the transition metal or metal or metalloid might be used. Within the meaning of the present application is that two transition metals or metals or metalloids might be mixed with each other. Also here, they can be present in there elemental form or as salt or as oxide. Thus, for example a metal oxide might be mixed with a metal, or a transition metal might be mixed with a metal, or a metalloid oxide might be mixed with a metal, or a metalloid might be mixed with a metal salt, or any other combination is within the meaning of the present application.

Mixtures within the meaning of the present application also encompasses that two or more transition metals or metals or metalloids might form another regulator compound. For example, zeolithes or any other alumosilicates can act as regulator within the meaning of the present application.

Preferably, the regulator is selected from an element of any of groups 7 to 12 of the periodic table, preferably of any of groups 8 to 11. Especially preferred are elements of groups 9, 10 or 11 of the periodic table. Especially preferred are Pt, Pd, Ir, Rh, Ru, Ag or mixture of these. In each of these preferred embodiments, the mentioned elements can be present in its elemental form or as salt or as oxide. Suitable salts are alkaline or earth alkaline metal salts. Thus, the salts and/or oxides of Pt, Pd, Ir, Rh, Ru, Ag are especially preferred.

Surprisingly it has been found that a regulator carrying free hydroxyl groups on its surface is especially preferred. Without being bound to theory such hydroxyl groups on the surface of a regulator might be able to activate the SO₃ or to stabilize any intermediates which form during the reaction between the alkane and SO₃. The hydroxy functionalization on the surface of the regulator thus enables a higher reactivity and the faster reaction to obtain alkane sulfonic acids, especially methane sulfonic acid.

In a preferred embodiment, the alkane sulfonic acid is methane sulfonic acid and thus, the alkane is methane. Preferably the present invention refers therefore to a process for the production of methane sulfonic acid in which methane and SO₃ are contacted with each other, preferably in the presence of an initiator.

Suitable regulators are for example alumina, especially γ-Al₂O₃, silica (SiO₂), zirconia, zeolithes, Pt, Pd, Ir, Rh, Ru, Ag, as well as mixtures of those. Especially alumina, silica, zirconia, zeolithes might provide for the free hydroxyl groups on the surfaces. Preferably, they also provide large surfaces for the reaction to take place. The surface might be provided in terms of the specific surface area according to the BET theory. The BET theory aims to explain the physical adsorption of gas molecules on a solid surface and serves as the basis for the measurement of the specific surface area.

In a preferred embodiment, alumina, especially γ-Al₂O₃, silica (SiO₂), zirconia or zeolithes may be used alone or together with another metal, especially together with any of Pt, Pd, Ir, Rh, Ru and/or Ag.

For example, γ-Al₂O₃ might be used together with Rh, whereas the Rh is located on the γ-Al₂O₃. Instead of the Rh also Pt and/or Ir and/or Ru and/or Ag might be used together with γ-Al₂O₃ The Rh or any of the other metals might, without being bound to that theory, activate the alkane, especially methane, for a faster reaction with the SO₃.

Surprisingly it has been found that the addition of a transition metal, or metal or metalloid in its elemental form enhances the yield of the reaction by means of improvement of selectivity. Adding the salts or the oxides of the transition metal or metal or metalloid leads to an enhancement of the reactivity resulting in a faster reaction. A faster reaction might be of interested in a commercial production when trying to transfer the batch process, which is common for the production of alkane sulfonic acids form alkane and SO₃, into a continuous process.

Thus, the process of the present invention and the inventive regulator enables the production of alkane sulfonic acids, especially methane sulfonic acid, from an alkane and SO₃ with a high yield, high selectivity and a high reactivity, whereas, depending on the individual necessity, selectivity and reactivity can be adapted independently of an adaption of the reaction condition or selected initiator.

The process according to the present invention takes place in a reactor. All components, namely the alkane, especially methane, SO₃, eventually needed solvents, initiators or acids, as well as the regulator of the present invention are all added into one reactor. Within the scope of the invention the regulator might also firstly be mixed with a liquid component, such as a solution comprising SO₃, and added in such combination to a reactor. Preferably, the regulator is directly added to the reactor without mixing it with any other component in advance.

The regulator according to the present invention can be used together with any initiator described for the production of alkane sulfonic acids from an alkane and SO₃, such as DMSP, Marshall's acid, Caro's acid, or the peroxo product as described in WO 2015/071455 A1. This enables the improvement of the initiator and balancing selectivity and reactivity of the reaction.

With the following examples the invention is further described without limiting it.

### EXAMPLES

### Example 1: Reaction without a Regulator

In a 3.75 L autoclave, 1000 g of 36% (w/w) oleum is charged, and the temperature controlled to 50° C. After a pressure of 100 bar of methane gas was set, intensive stirring is performed with a stirrer from the company Parr. Now, the initiator solution consisting of 96 ml methanesulfonic acid and 3.6 ml hydrogen peroxide (70%) is metered dropwise to the solution. The pressure drops to 36 bar within 5 hours. The yield is higher than 90%, based on sulfur trioxide. The reaction product contains 42% (w/w) methanesulfonic acid.

### Example 2: Reaction with gamma-Aluminium oxide as regulator

In a 3.75 L autoclave, 1 g of gamma-Aluminium oxide and 1000 g of 36% (w/w) oleum is charged, and the temperature controlled to 50° C. After a pressure of 100 bar of methane gas was set, intensive stirring is performed with a stirrer from the company Parr. Now, the initiator solution consisting of 96 ml methanesulfonic acid and 3.6 ml hydrogen peroxide (70%) is metered dropwise to the solution. The pressure drops to 28 bar within 3.5 hours. The yield is higher than 95%, based on sulfur trioxide. The reaction product contains 45% (w/w) methanesulfonic acid.

### Example 3: Reaction with Rhodium Chloride as Regulator

In a 3.75 L autoclave, 1 g of rhodium chloride and 1000 g of 36% (w/w) oleum is charged, and the temperature controlled to 50° C. After a pressure of 100 bar of methane gas was set, intensive stirring is performed with a stirrer from the company Parr. Now, the initiator solution consisting of 96 ml methanesulfonic acid and 4 ml hydrogen peroxide (70%) is metered dropwise to the solution. The pressure drops to 35 bar within 3 hours. The yield is higher than 90%, based on sulfur trioxide. The reaction product contains 43% (w/w) methanesulfonic acid.

1. A process for the production of alkane sulfonic acids, comprising contacting an alkane and SO₃ with each other in the presence of a regulator, said regulator being a transition metal or metal or metalloid of any of groups 3 to 14 of the periodic table, or mixtures of two or more of those, the transition metal or metal or metalloid being present in its elemental form, as a salt, or as an oxide.

2. The process according to claim 1, wherein the regulator is one or more transition metals.

3. The process according to claim 1, wherein the regulator is one or more transition metals of group 9, 10 or 11 of the periodic table.

4. The process according to claim 1, wherein the regulator has free hydroxyl-groups on its surface.

5. The process according to claim 1, wherein the regulator is present as a salt.

6. The process according to claim 1, wherein the regulator is selected from the group consisting of alumina, silica (SiO₂), zirconia, a zeolite, Pt, Pd, Ir, Rh, Ru, Ag and mixtures of those.

7. The process according to claim 6, wherein alumina, silica, zirconia or silica are used as regulator in combination with at least one metal selected from the group consisting of Pt, Pd, Ir, Rh, Ru and Ag.

8. The process according to claim 1, wherein the alkane is methane and the alkane sulfonic acid is methane sulfonic acid (MSA).

9-10. (canceled)
